(19) United States
(12) Patent Application Publication
(10) Pub. No.: US 2026/0345025 A1
(43) Pub. Date: Nov. 5, 2026

(54) AN ENDOSCOPE ACCESSORY

(71) Applicant: MIWENDO SOLUTIONS, S.L., BARCELONA (ES)

(72) Inventors: MARTA GUARDIOLA GARCÍA, BARCELONA (ES); ROBERTO SONT COLOMER, BARCELONA (ES); MARIA GLÒRIA FERNÁNDEZ ESPARRACH, BARCELONA (ES); IGNASI BELDA REIG, BARCELONA (ES)

(21) Appl. No.: 19/099,271

(22) PCT Filed: Jul. 28, 2023
(86) PCT No.: PCT/EP2023/071041

§ 371 Date: Jan. 28, 2025

(30) Foreign Application Priority Data
Jul. 29, 2022 (EP) 22382732.0

(51) Int. Cl. A61B 5/0507; A61B 5/00

(52) U.S. Cl. CPC A61B 5/0507; A61B 5/4255; A61B 5/6852; A61B 2562/0228; A61B 2562/12

(57) ABSTRACT

An endoscope accessory with: a first flexible substrate including a first side and a second side, wherein the first flexible substrate further includes a window defining an opening between the first side of the first flexible substrate and the second side of the first flexible substrate. The endoscope accessory further includes a second substrate comprising a first surface and a second surface. The first surface of the second substrate includes at least one radiating element which is coupled to a transmission line. The second substrate is attached to the first flexible substrate with the first surface of the second substrate adjacent to the first flexible substrate. The radiating element is at least partially aligned with the window of the first flexible substrate. Some examples describe slot antennas printed or etched on a rigid substrate and SIW (Substrate Integrated Waveguide) type slot antennas.

### BACKGROUND

The present disclosure is directed to the field of medical systems for detecting diseases. In particular, the disclosure relates to a system for prevention and diagnosis of diseases such as cancers, for example colorectal cancers, among other pathologies.

Most cancers arise from precursor lesions, for example colorectal cancers arise from adenomatous polyps according to the adenoma-carcinoma sequence. In recent years there have been many improvements in image quality provided by currently available endoscopes, HD endoscopes, complementary functions such as electronic chromoendoscopy, multi-lens colonoscopes and image magnification. The use of these resources makes scanning laborious and requires specific training for the endoscopist. Due to said limitations of conventional endoscopy, more innovative complementary techniques such as computer vision have appeared. Such techniques are based on automatically processing the colonoscopy image and most of the studies are based on the description of morphological characteristics such as shape or texture to determine the existence of a polyp.

Another non-invasive emerging technique is microwave imaging. This technique does not rely on optical images. Microwave signals penetrate light opaque materials and operate a new contrast mechanism based on dielectric properties of tissues. Each tissue has its own dielectric properties that vary according to their conditions (hypoxia, ischemia, neoplasm, etc.). Microwave imaging systems can sense the dielectric properties of the human body and obtain an image representing the spatial distribution of tissue's pathological conditions without requiring direct contact. Microwaves are applied in the field of medicine since the 1980s for both treatment (hyperthermia and ablation) and diagnosis. Breast cancer and cerebral hemorrhage detection are the most well-known applications. The main advantages of microwaves are safety (non-ionizing and low power radiation) and low cost, making microwaves a powerful technique for monitoring and prevention.

Microwave imaging systems can provide information in many endoscopic or catheter-based mapping applications.

At present, systems based on microwaves have been designed to provide compact assemblies.

It is an object of the present disclosure to provide systems and devices based on microwave technology for prevention and diagnosis of diseases such as cancers (e.g. colorectal cancers, among other types of cancers) whose compact design allows integrability.

### SUMMARY

In a first aspect of the disclosure, there is provided endoscope accessory, the accessory comprising:
a first flexible substrate comprising a first side and a second side, wherein the first flexible substrate further comprises a window the window defining an opening between the first side of the first flexible substrate and the second side of the first flexible substrate;
a second substrate comprising a first surface and a second surface, the first surface of the second substrate comprising at least one radiating element, the radiating element coupled to a transmission line, the transmission line connected to a connector; wherein the transmission line is configured to transmit electrical signals, and the electromagnetic waves associated therein, towards the radiating element;
wherein the second substrate is attached to the first flexible substrate with the first surface of the second substrate adjacent to the first flexible substrate; and wherein the radiating element is at least partially aligned with the window of the first flexible substrate.

In a second aspect of the disclosure, there is provided a system to detect polyps, the system comprising an accessory according to the disclosure and an external unit operable to generate, receive and process microwave signals and operable to activate an alert if a polyp is detected.

In a third aspect, there is provided a method of manufacture of an endoscope accessory according to the disclosure, the method comprising the steps of: providing a first flexible substrate; cutting at least one window on the first flexible substrate, the window defining an opening between a first side of the first flexible substrate and a second side of the first flexible substrate; including at least a radiating element on the second substrate; attaching or soldering at least one second substrate to the first flexible substrate, the second substrate comprising the at least one radiating element, in such a way that the at least one radiating element is at least partially aligned with the at least one window; coupling the radiating element to a transmission line; and connecting the transmission line to a connector.

### BRIEF DESCRIPTION OF THE DRAWINGS

Examples will now be described, by way of non-limiting example, with reference to the accompanying drawings, in which:

FIG. 1 an accessory for an endoscope.

FIG. 2 shows an accessory according to the present disclosure.

FIG. 3 shows an accessory according to the present disclosure.

FIG. 4 shows an accessory according to the present disclosure.

FIG. 5 shows an accessory according to the present disclosure.

FIG. 6A shows an accessory according to the present disclosure.

FIG. 6B shows an accessory according to the present disclosure.

FIG. 7A shows a second substrate for an accessory.

FIG. 7B shows a second substrate for an accessory according to the present disclosure.

FIG. 8 shows a system according to the present disclosure.

FIG. 9 shows an accessory according to the present disclosure, an outer casing and an inner casing.

FIG. 10 shows a system according to the present disclosure.

### DETAILED DESCRIPTION

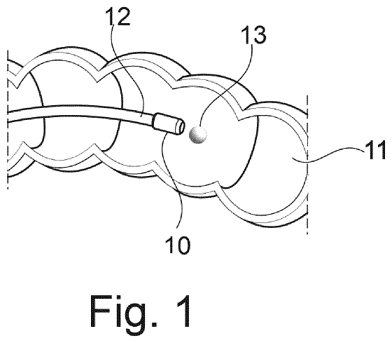

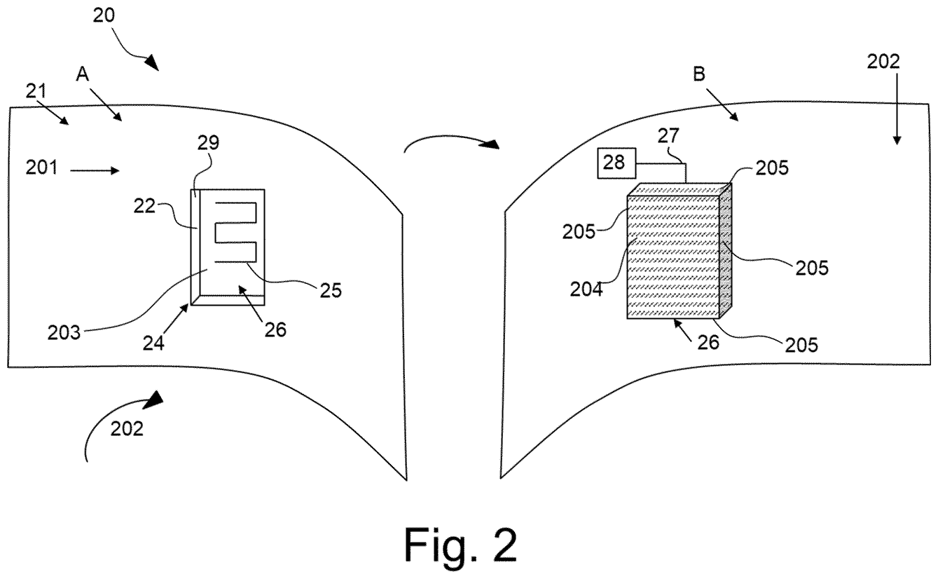

The present disclosure relates to an accessory. FIG. 1 shows an accessory 10 for an endoscope 12 where the endoscope 12 is inserted into a colon 11 presenting a polyp 13. An accessory 10 may be compact so that the usual dimensions of an endoscope are not compromised or are compromised in a minimal range of tolerance. An endoscope 12 is to be understood as a long, thin, flexible tube that has a light and one or more cameras at one end. FIG. 2 shows an accessory 20 for an endoscope, or endoscope accessory according to the present disclosure.

The accessory 20 comprises, as seen in FIG. 2, a first flexible substrate 21 configured to be wrapped around a part of an endoscope tube. Configured to be wrapped around a part of an endoscope tube comprises offering a malleability or flexibility allowing to accommodate the first flexible substrate on a part of a surface of an endoscope tube at least partially or allowing the substrate to cover a part of a surface of an endoscope tube at least partially. The part of the surface of the endoscope tube may be the end towards which a light and a camera are comprised; preferably, in use, the camera and the light are not covered by the accessory wrapping the endoscope tube. The first flexible substrate 21 may present a thickness below 0.2 mm, present a loss tangent below 0.004, and may be made of dielectric material. The first flexible substrate 21 may be at least partially metallized. In the present disclosure, metallized may be understood as at least partially covered by a metal material or at least partially covered by a metal layer. The first flexible substrate 21 may be covered by copper on the first side 201 and on the second side 202. The copper thickness may be 18 um or less and may present a surface roughness on the side of the dielectric around 2.8 um RMS roughness. The first flexible substrate 21 may comprise a board of flexible substrate which may present dimensions suitable to be wrapped around a part of a surface of an endoscope tube at least partially.

The first flexible substrate 21 of the accessory 20 comprises a first side 201 as seen in the representation indicated by the letter A in FIG. 2, and a second side 202, where the second side 202 is represented on the right-hand side of FIG. 2 as seen in the representation indicated by the letter B. The side B is visible after flipping over the accessory as seen in first side A. In the present disclosure, the second side 202 may also be referred to as the inner side, since it is the side which, in use, would face or would touch an endoscope when the accessory wraps around a part of a surface of an endoscope tube at least partially. In the present disclosure, the first side 201 may also be referred to as the outer side, since it is the side which, in use, would face a colon tissue when the accessory wraps around a part of a surface of an endoscope tube at least partially. The first flexible substrate 21 further comprises a window 24, the window defining an opening or hole between the first side 201 of the first flexible substrate 21 and the second side 202 of the first flexible substrate.

The accessory 20 further comprises a second substrate 26 comprising a first surface 203 and a second surface 204, the first surface 203 of the second substrate 26 comprising at least one radiating element 25, the radiating element coupled to a transmission line 27. The second substrate may be made of a dielectric material, for example including ceramics, glass or plastic. A radiating element 25 may be defined as a unit within an antenna which in itself is capable of radiating or receiving radiofrequency energy. In the present description, an antenna may be formed by the radiating element 25 or an antenna may be formed by the radiating element 25 and additional elements. The assembly of the second substrate 26 and the radiating element 25 may form an antenna. The second substrate 26 is attached to the first flexible substrate 21 with the first surface 203 of the second substrate adjacent to the inner side of the first flexible substrate 21, and the radiating element 25 is aligned with—i.e. coincides, matches, or fits, or corresponds to,-the window 24 of the first flexible substrate 21, in such a way that any radiation performed by the radiating element is allowed to pass through the window 24. The radiating element is coupled to the transmission line 27 for receiving a signal to be radiated and/or for transmitting over the transmission line 27 a received signal. In the present description the radiating element may be a metallic element, or a patch, or a slot, or an etched element on a metallized first surface 203 of the second substrate 26, or any kind of printed radiating element. Coupled to the transmission line 27 comprises in electrical communication or in electrical connection. The transmission line 27 or several transmission lines may be comprised on the first flexible substrate 21. In examples where the first flexible substrate is at least partially metallized, the transmission line(s) may be etched on the metallic layer or metal material. In examples, the transmission line(s) may be a printed metallic line(s) on the first flexible substrate. The radiating element may be printed on the second substrate. Printed on the second substrate may comprise a metallic line printed on a second substrate made of a dielectric material. Alternatively printed on the second substrate may comprise a line or a slot etched on a metallized—or at least partially metallized—second substrate. Printed or printing may comprise techniques such as laser-printing, and/or photolithography, and/or screen-printing.

In FIG. 2, the radiating element 25 is on the first surface 203 of the second substrate 26. The transmission line 27 or feeding line is connected to a connector 28. The transmission line 27 may be or may be part of a printed circuit over which electrical signals, and associated electromagnetic, EM, wave, may be transmitted. The electrical signals may be transmitted over the transmission line 27—also referred to as printed circuit—towards the radiating element 25. The transmission line 27—or printed circuit—may be comprised on either side of the first flexible substrate 21. A ground plane may be comprised on an opposite side. In examples, the first side 201—or outer side—of the first flexible substrate 21 comprises the transmission line 27—or printed circuit—and the second side 202—or inner side—comprises the ground plane not shown in FIG. 2. In examples, the first side 201—or outer side—of the first flexible substrate 21 comprises a ground plane not shown in FIG. 2 and the second side 202—or inner side—of the first flexible substrate 21 comprises the transmission line 27—or printed circuit—. One of the advantages of an accessory comprising a ground plane on the first side 201—outer side—of the first flexible substrate 21 is that, when wrapped around an endoscope tube and in operation, any interference between any radiation emitted by the transmission line(s)—or printed circuit—and the radiation emitted by the radiating element(s) or antenna(s) is avoided. The transmission line 27 may be printed on the second side 202 of the first flexible substrate 21 and may be joint, preferably by welding techniques, to a first surface 203 of the second substrate 26 in order to transmit and receive signals using the radiating element 25 or antenna, sending and/or receiving the signals through the transmission line 27.

The second substrate 26 is attached to the first flexible substrate 21 with the first surface 203 of the second substrate 26 adjacent to the first flexible substrate 21, in particular, adjacent to or adjoining the second side 202 of the first flexible substrate 21. The terms attached to and adjacent may comprise, in the present disclosure, in contact. In some examples the first flexible substrate 21 and the second substrate 26 are attached by soldering techniques. The terms attached to and adjacent may comprise, in the present disclosure, at least partially in contact. The terms attached to and adjacent may comprise, in the present disclosure, separated by a layer of a different material, for example, adhesive material. Advantageously having a first substrate attached and in-contact with a second substrate allows reducing at least one dimension of the accessory.

The radiating element 25 or antenna is aligned with—i.e. matches, or fits, or corresponds, or coincides—with the window 24 of the first flexible substrate 21 in such a way that, in use, when the accessory 20 is wrapped around a part of an endoscope tube and in operation, the radiating element 25 or antenna is exposed through the window 24 and radiates outwards—i.e. towards an opposite side to the inner side of the first flexible substrate 21. In other words, in such a way that any radiation performed by the radiating element is allowed to pass through the window 24. FIG. 2 shows a rectangle-formed window 24, but the window may adopt different forms, such as a circle, or an oval, or a pentagon, or a hexagon or a square.

As previously described, the first surface 203 of the second substrate 26 comprises the radiating element 25. The first surface 203 faces outwards when, in use, the accessory wraps an endoscope tube. In such configuration the radiating element 25 or antenna radiates, i.e.: transmits and receives, outwards from the endoscope tube. As it may be understood, the dimensions of the first surface 203 of the second substrate 26 may not coincide with the dimensions of the window 24; for example, the first surface 203 of the second substrate 26 may present larger height and/or width than the height and/or width of the window 24. The radiating element 25 is comprised within the window, which means that the effective height and the effective width of the radiating element 25 are below the height and below the width of the window 24, as the figures show. The accessory described in the present description may be used in microwave imaging practices. The radiating element may transmit a signal within a colon and/or the radiating element may receive a signal reflected by the colon to provide an image which can be analyzed to detect or diagnose, for example, the existence of a polyp. The accessory as described allows transmitting a microwave signal into the inner part of a colon and allows receiving a reflected microwave signal. The transmitted and received signals may be compared or analyzed to detect any anomaly. A single radiating element or a single antenna may be used for transmission and reception by the use of, for example, a circulator.

In some examples, the second substrate 26 is metallized, comprising metallized first surface 203, metallized second surface 204 and metallized second edges 205, and at least an edge 29 of the window 24, for example one edge 29 or for example four edges or the window 24, comprises a metallized portion 22. The edge 29 of the window may be defined as an edge defining the window and which traverses the first flexible substrate 21 from the first side 201 to the second side 202 or vice-versa. In some examples one edge 29 of the window 24 comprises a metallized portion. In some examples, two or more or all edges of the window are metallized or covered by a metallic layer. The metallization of at least an edge of the window allows an electrical coupling between the first side 201 and the second side 202 of the first flexible substrate 21. Such electrical coupling avoids any radiation from being transmitted into the dielectric of the first flexible substrate in operation. The metallization provides efficiency to the radiation of the radiating element or antenna. The thickness of the window is the thickness of the first flexible substrate. In the examples where the first side 201 and the second side 202 of the first flexible substrate 21 are metallized, and an edge 29 of the window 24 comprises a metallized portion, the first side 201 and the second side 202 of the first flexible substrate 21 are interconnected such that no electrical field can propagate inside the first flexible substrate 21, which would affect the radiation during operation.

In some examples, the metallized second edge 205 or second edges 205 of the second substrate 26 may function as a waveguide to the signal transmitted and/or received by the radiating element 25 or antenna. The waveguide may be formed by the metallized second edges 205 of the second substrate 26 and the metallized surfaces 204 and 203 of the second substrate 26. Such configuration may correspond to a substrate integrated waveguide configuration of the antenna or radiating element of the accessory 20. In some examples the edge of the second substrate 26 comprises a non-metallized portion aligned with the transmission line 27 such that an electrical bridge is avoided between the waveguide and the transmission line 27. Otherwise, the edge would be shortcut with the transmission line 27.

The accessory may further comprise an array of multiplexed antennas that transmit and receive microwave signals that interact with the dielectric properties of the colon tissues during colonoscopy, in order to sense tissue distribution and detect possible pathological conditions on any part of the perimeter of the colon. In examples, the accessory comprises two or more reception radiating elements—or antennas—, and two or more transmission radiating elements—or antennas. In such a case, a first multiplexor may be used for selecting one transmission radiating element and a second multiplexor may be used for selecting one reception radiating element. A transmission signal transmitted by the transmission radiating element and a reception signal received by the reception radiating element may be processed using an algorithm, such that dielectric properties of the colon may be obtained. Processing of the transmission and/or the reception signals may comprise quantitative reconstruction of the dielectric properties of the colon and/or a reconstruction of a contrast in dielectric properties. The multiplexors may select radiating elements or antennas sequentially and cyclically so that a 360° microwave image of the colon tissue can be obtained. The accessory may comprise the multiplexors, or the multiplexors may be outside the accessory, for example in an external unit, as explained further below.

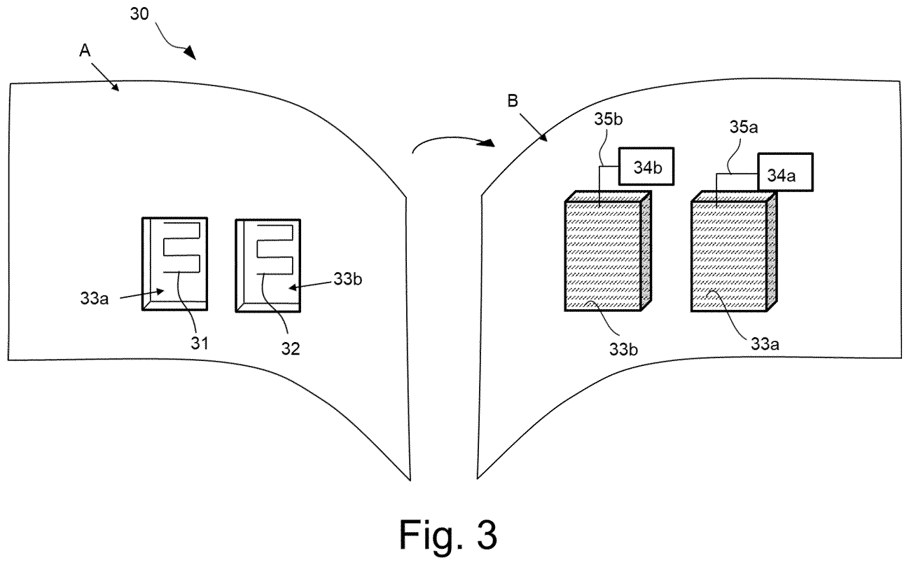

FIG. 3 represents a first side A of an example accessory 30 and a second side B—or inner side B—of the example accessory 30. The side B is visible after flipping over the accessory as seen in first side A. In some examples as the one in FIG. 3, the accessory 30 comprises at least two radiating elements 31, 32 comprised on second substrate 33a and second substrate 33b. The assembly of the second substrate with each one of the radiating element 31 and the radiating element 32 form either antenna, wherein a first antenna is a reception antenna, and a second antenna is a transmission antenna. The side B of the example accessory 30 shows a first connector 34a and a second connector 34b. In this case, the radiating element 31 is in connection to the first connector 34a through a first transmission line 35a and the radiating element 32 is in connection to the second connector 34b through a second transmission line 35b. Any further radiating element may be in connection to a further connector through a further transmission line. Other examples explained hereinbelow, provide a different configuration with multiplexors, MUX, which may avoid having one connector per antenna or per radiating element, as in the case of the example of FIG. 3.

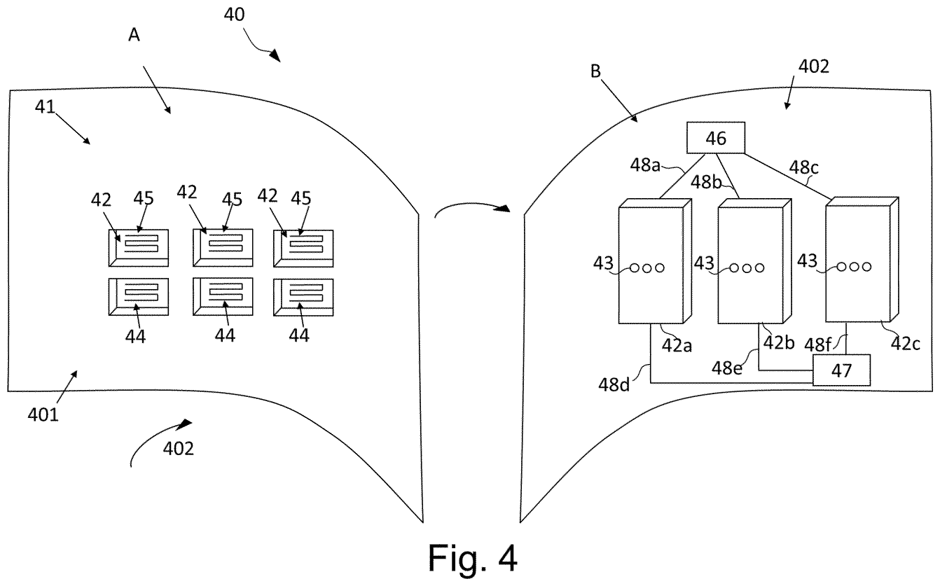

In some examples, the antennas are meandered slot antennas, wherein the radiating element is a meander etched on the second substrate. FIG. 4 represents an example of accessory 40 with meandered slot antennas. The accessory 40 comprises 6 meander-shaped radiating elements forming part of 3 reception antennas 44 and 3 transmission antennas 45. FIG. 4 shows the first flexible substrate 41, and 3 second substrates 42a, 42b, 42c, also referred to as 42. Each pair of transmission and reception antenna is comprised on each of the 3 second substrates substrate 42a, 42b, 42c. One or more of the second substrates 42a, 42b, 42c comprise a plurality of metallized via-holes 43 in between each radiating element at each side of the via-holes. In the example shown, either radiating element may be a reception antenna 44 and a transmission antenna 45. The via-holes 43 act as a metallic wall and allow to constrain the electromagnetic field inside the dielectric of the second substrate corresponding to each radiating element, avoiding therefore interferences between the transmission and reception electromagnetic fields. The via-holes 43 may be placed substantially in the middle of the second substrates 42a, 42b, 42c at a same distance from the transmission and reception radiating elements—or antennas—. In other examples the via-holes 43 may be placed at a first distance from one end of the second substrates 42 and a second distance from the other end of the second substrates 42. Having an equidistant line of via-holes 43 may provide uniform behavior of transmission and reception radiating elements. The via-holes 43 may comprise a diameter comprised in a range of, for example, [0.1-1] mm, and preferably the via-holes 43 present a 0,6 mm diameter. The via-holes 43 may be metallized or covered with a metallic layer. The transmission antennas 45 of FIG. 4 are coupled to a first multiplexor 46 and the reception antennas 44 are coupled to a second multiplexor 47. The antennas may be coupled through respective transmission lines 48a . . . 48f to the multiplexor or multiplexors for the selection of a signal coming to/from one of the three antennas of FIG. 4. This configuration with multiplexors, MUX, may avoid having one connector per antenna or per radiating element, as in the case of the example of FIG. 3.

FIG. 4 represents, in the representation indicated by the letter A, the external part of the accessory showing the antennas through the windows. As seen, the external part represented in A, does not show the via-holes 43 as they hide behind the first side 401 of the first flexible substrate 41. The via-holes 43 are visible from the inner part representation B, showing the second substrates 42 on the second side 402 of the first flexible substrate 41. The via-holes 43 traverse the second flexible substrates 42.

The frequency of emission of the transmission and reception antennas may be comprised within 1 GHz and 20 GHz. As known, microwaves antennas emit waves whose wavelengths, λ, wherein λ is defined as “propagation speed/frequency” range from about one meter to one millimeter corresponding to frequencies between 300 MHz and 300 GHz. The frequency of emission may be around 7.5 GHz for the application of the accessory of the present disclosure. The size of the radiating elements, which in the example of FIG. 4 are meander slots, may vary depending on the diameter of the endoscope wrapped by the accessory. Some endoscopes may present a 13 mm diameter. Advantageously, antennas separated λ/2 or less are used for imaging purposes.

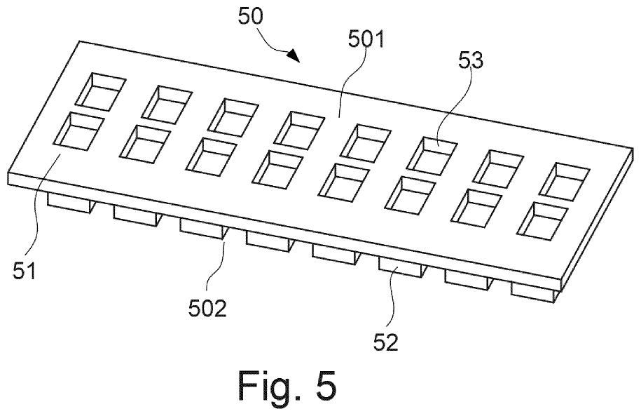

FIG. 5 represents a three-dimensional view of an example accessory 50 according to some examples of the present disclosure. The accessory 50 comprises a first flexible substrate 51 comprising a first side 501 and a second side 502. The first flexible substrate 51 comprises windows 53 discovering at least part of second substrates 52 from the second side 502. FIG. 5 shows 16 windows 53 which, each, comprise 1 radiating element—not shown—, forming 16 antennas. In this example, 8 of such 16 antennas 53 may be reception antennas and 8 may be transmission antennas. Building 8 pairs of antennas provides a good balance between a microwave image resolution obtained and a size of the antennas. The window 24 of the accessory of FIG. 5 may present a length varying in a range from 0.1 λ to 0.5 λ, where λ represents the length of a wavelength of a microwave signal which, in use, is transmitted and/or received by the accessory. In an example, the window 24 presents a length of 6.4 millimeters (mm) being, substantially, 0.16 λ. The window 24 may, for example, present a width varying in a range from 0.1 λ to 0.5 λ, or for example, may present a width of 4.8 mm or 0.12 λ. The window 24 may present a thickness varying in a range from 0.01 λ to 0.05 λ, or for example, may present a thickness of 0.81 mm or 0.02 λ. Other configurations or combinations of transmitting and/or antennas are possible. In use the accessory wraps an endoscope tube, and each radiating element or antenna radiates so that corresponding emission beams flourish from each antenna covering a 360° field of vision to ensure the visualization of the full perimeter of the colon.

In some examples the antenna is etched on the first side of a second substrate. In the cases where the second substrate comprises a dielectric and is covered with a metallic layer, the etched antenna is formed by eliminating the metallic layer and leaving the dielectric visible. The electromagnetic field is propagated within the dielectric and is used to transmit and receive signals by the antenna. In such examples, and if the antenna is a meandered antenna, a meander is etched on the second substrate. The second substrate may comprise a board made of dielectric, or some type of high frequency circuit materials, such as glass reinforced hydrocarbon/ceramic laminates.

In some examples the second side of the first flexible substrate is electrically coupled to a ground plane of the antenna by means of an edge platting of at least a portion of the edge of the window.

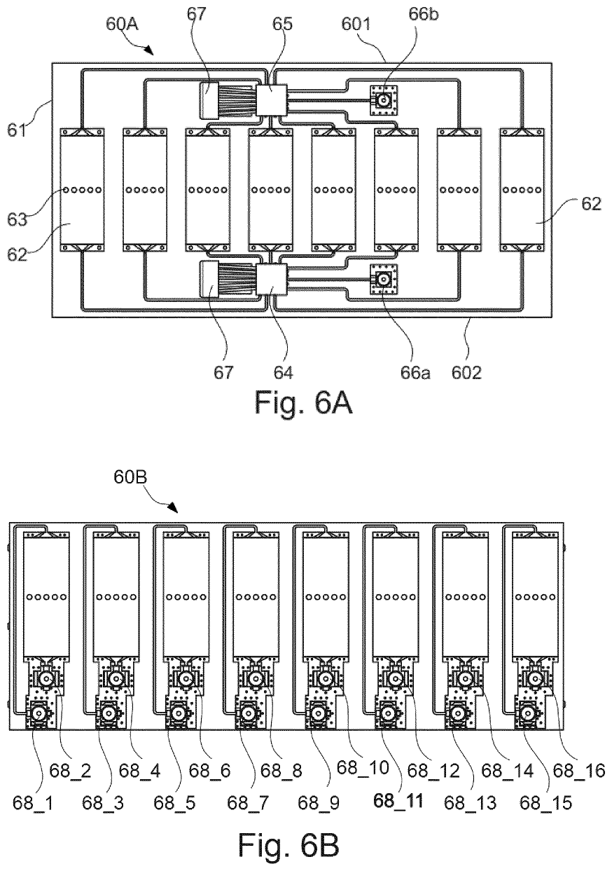

FIG. 6A shows an accessory 60A. More precisely, FIG. 6 shows a second side 602 of a first flexible substrate 61, wherein the second substrates 62 are placed or mounted. The second substrates 62 comprise via-holes 63. The first flexible substrate 61 comprises, on the second side 602, a first multiplexor 64 and a second multiplexor 65. The multiplexors 64 and 65 receive, via corresponding transmission lines or microstrips, the signals from the antennas in the second substrates 62. As seen, the transmission lines or microstrips are connected to a connector 66a or 66b through either multiplexor 64 or 65. In some examples, the multiplexor, MUX, 64 receives signals from reception antennas and the MUX 65 transmits signals to transmission antennas. In other examples other configurations or operating modes are possible. The selection of the signal which is transmitted towards and from a MUX (64, 65) from and towards a connector 66, for example a radio frequency connector 66, may be indicated by an external unit connected to a multiple connector 67. The radio frequency connector 66 may be used to connect the accessory to an external unit (not shown in FIG. 6A). FIG. 6B shows an accessory 60B comprising a connector 68 per antenna. The accessory 60B comprises a total of 16 connectors, referenced as 68_1 to 68_16 in the FIG. 6B. In the example of FIG. 6B the MUXs are not comprised in the accessory 60B, in which case a radio frequency signal may be sent from the accessory to one or more MUX outside the accessory. In the example of FIG. 6B, a pair of multiplexors may be comprised in an external unit for the selection of antennas.

The transmission lines may comprise transmission lines for transmitting radio frequency signals and transmission lines for transmitting control signals for the MUX.

As seen, some of the examples describe slot antennas printed or etched on a rigid substrate. More specifically, SIW (Substrate Integrated Waveguide) type slot antennas substantially reduce manufacturing costs. Some of the examples describe meandering slot antennas. Some examples include SIW (Substrate Integrated Waveguide) type slot antennas built on negative order resonance for example n=−1 which miniaturize the overall size of the accessory. SIW technology is based on using the antenna substrate itself to create the waveguide.

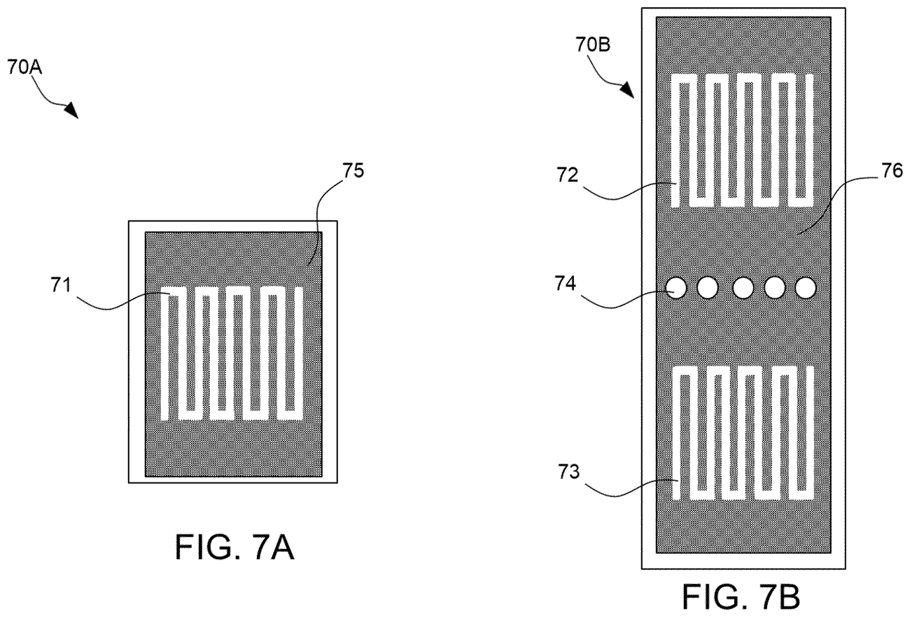

FIGS. 7A and 7B show two variants in which the antennas may be comprised in the second substrate. FIG. 7A shows a second substrate 70A comprising a metallized layer shown as the grey zone 75. The metallized layer may be a layer of copper covering the substrate. The substrate may be a dielectric. The grey zone 75 is etched letting a meandered part of the dielectric show, represented as the white meander 71. The meandered part of the dielectric shown is a radiating element which may be or may be part of an antenna. The electromagnetic field of the signals propagated by the antenna propagate through the dielectric. FIG. 7B shows a second substrate 70B comprising a metallized layer shown as the grey zone 76. The metallized layer may be a layer of copper covering the substrate. The substrate may be a dielectric. The grey zone 76 is etched letting two meandered parts 72, 73 of the dielectric show, represented as the white meanders 72 and 73. The meandered parts of the dielectric shown are two radiating elements which may be or may be part of an antenna or two antennas. The second substrate 70B is drilled so that the second substrate 70B comprises via-holes 74. In some examples, the radiating elements may form a multiplexed circular array of antennas able to cover the full perimeter—or 360°—of the colon when the endoscope accessory wraps an endoscope. Each radiating element may be configured to unidirectionally radiate towards the colon wall, radiating at a frequency in the range 5-8 GHz which satisfies providing enough resolution and contrast between the colon mucosa and the polyps. The radiating elements may form a low cost, multiplexed circular array of electrically small antennas. As seen, the endoscope accessory allows for a device which is configured to radiate signals or microwave signals via one or more of transmission radiating elements or transmission antennas, towards the mucosa or inner or innermost layer of a colon and configured to receive a reflected signal, the signal being scattered or reflected by the colon, via one or more of reception radiating elements or reception antennas. The final dimensions of the endoscope accessory or acquisition device may be between 28 and 33 mm in length, preferably 30 mm in length and may be between 15 mm and 25 mm, preferably 20 mm in diameter, having a total thickness between 2 mm and 5 mm, preferably a thickness of 3 mm. The dimensions and shape of the device ensure non-obstruction of the front tip of a colonoscope, avoids camera concealment, injuring the patient or hindering the maneuverability of the colonoscope.

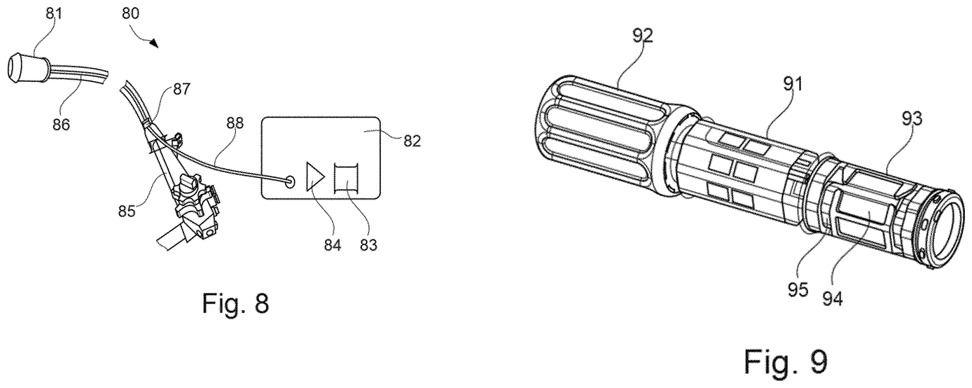

In some examples the accessory may form part of a system for detecting polyps, as seen in FIG. 8. The system 80 may comprise: an accessory 81 of any of the examples of the present disclosure and an external unit 82. The external unit 82 may comprise a processing unit 83 configured to generate, transmit and receive microwave signals towards and from the accessory 81 and configured to activate an alert if a polyp is detected.

In some examples, the system 80 may comprise one or more multiplexors 84 for the selection, reception and/or transmission signals, or for the selection of a signal emitted by one of the antennas provided in the accessory 81.

In some examples, the multiplexor(s) is(are) provided in the accessory. In some examples the external unit 82 comprises one or more multiplexors 84. The external unit 82 may, in some examples, generate signals governing the multiplexers and control which pair of transmission and reception antennas should activate.

FIG. 8 shows a medical system 80 based on microwave imaging for polyp detection. One of the example accessories 81 disclosed herein is attached to a distal end of a colonoscope 85. The medical system 80 improves prevention and early detection of colorectal cancer by detecting polyps, which may be precancerous lesions. The accessory 81 is connected via cables to the external unit 82. The external unit comprises a processing and generating signal unit 83. As shown in FIG. 8, the accessory 81 may be cylindrical and configured to be attached to a conventional colonoscope 85 by way of the cylindrical shape. The accessory 81 may be connected to the external unit 82 via cable. FIG. 8 shows an example system 80 in which different transmission lines are used for two differentiated parts of the system 80. The accessory 81 may further comprise a first transmission line 86. The accessory comprising the first transmission line 86 may be connected (for example from the connector 66 shown in FIG. 6A) to a connection point 87 and a second transmission line 88 may connect the connection point 87 to the external unit 82. The advantage of having different transmission lines used for two differentiated parts of the system is that the first part comprising the accessory 81 further comprising the first transmission line 86 may be disposable or not reusable, increasing the hygienic security of a process for detecting polyps. In the case where the second part comprises the connection point 87, the colonoscope and the second transmission line 88 may be reusable or not disposable. In some examples the intermediate connector 87 comprises one or more multiplexors. Advantageously, in cases where the first part comprising the accessory 81 and the first transmission line 86 is disposable and where the MUX are not comprised in the accessory, the intermediate connector 87 comprising the MUX provides the advantage that from the intermediate connector 87, 3 wires, compared to 16, are needed transmit the signals towards an external unit from the intermediate connector 87.

The accessory 81 may comprise a set of antennas that transmit and receive microwave signals that interact with the dielectric properties of the colon tissues while a colonoscopy is being performed. The signals from the set of antennas may be multiplexed by multiplexors. The accessory 81 may comprise one or more multiplexors. The external unit 82 may comprise the multiplexors instead of the accessory 81. In a case where the accessory comprises no multiplexors, the accessory may comprise one connector per antenna or per radiating element—as seen in FIG. 6B—that are connected to corresponding transmission lines. The accessory may contain 16 connectors—as seen in FIG. 6B—that are connected to 16 transmission lines or cables—not shown—. The 16 transmission lines may be connected to the external unit 82 or to the connection point 87.

The external unit 82 may comprise a set of coaxial cables connected to the accessory 81 conducting microwave signals to the accessory. According to an example, the coaxial cables of the set of coaxial cables may present a length of approximately 400 cm. For the transmission of 7,5 GHz microwave signals, coaxial cables presenting a minimum diameter of 1.13 mm may be used. Connectors for coaxial cables may comprise miniature IPEX MHF4L and/or SMA connectors and/or Samtec CONN SOCKET 40POS SMD GOLD. Optical fiber may also be used as a transmission line, although any wireless communication technique may also be used between the antennas and the external unit. Planar transmission lines such as microstrip or stripline type lines may also be used.

A signal emitted by the antennas may reach the external unit 82 via one or more transmission lines and the processing unit or CPU 83 located in the external unit 82 may process such signal by means of specific algorithms. The processing may be performed in real time. An acoustic signal may be emitted when the algorithm indicates the presence of a polyp, although other types of alerts may be comprised, such as, for example visual alerts, communication alerts via wireless communication, alerts on a smartwatch or wearable, etc. The system 80 automates the detection of polyps by producing an alert when a polyp is detected and may increase the field of view to 360° when the antennas surround the distal end of the colonoscope 85 in a 360° range. The system 80 may allow, depending on the algorithms implemented by the CPU 83, differentiating different types of polyps from healthy colon mucosa, and indicate the position of a polyp without changing current clinical practices.

The external unit may comprise one or more of the following elements:
an antenna array control system;
a hardware, or radio frequency RF hardware, module for generating and receiving the microwave signal;
other RF components such as amplifiers or directional couplers to condition the microwave signal;
a central processing unit (CPU) with a real-time operating system, wherein the operating system may execute acquisition algorithms that may communicate with the RF circuit, signal processing algorithms (that may include calibration, imaging and detection), data management (that may store data, may delete data, may control access and may manage errors), system configuration, system update, and graphical interface;
a housing with a USB port for software update (such as system update) or store data, power on/off button and a screen to facilitate the interaction between the endoscopist and the system;
a cabling and interface for connection to the accessory;
a buzzer to generate the acoustic signal;
a power supply.

In the present description the hardware or RF hardware module may be understood as a device which may apply a stimulus wave to the accessory and perform a series of measurements and calculations. A two-port hardware module can measure both the reflected signals from the accessory and the transmitted signals to the accessory. Additionally, the hardware module can calculate S-parameters and other related parameters for the accessory, where S-parameters describe an input-output relationship between the two ports in the hardware module. The hardware module can repeat this procedure using different frequencies and/or power levels to measure the desired characteristics of the accessory or of a colon during operation.

The hardware or RF hardware module may be comprised either in the external unit or in the accessory of the present disclosure and comprise an integrated hardware module for generating and receiving the microwave signal, for example a chip, or a vector network analyzer (VNA) or an ultrawideband sensor. The external unit may be powered through a medical grade AC/DC adapter connected to an electrical grid as a power supply. The dimensions of the external unit may be equal or less than 48×32×16 cm.

The antenna array control system may generate a sequence of control signals to control the multiplexers that are responsible for selecting transmitting and receiving signals to or from the antennas. The control system may select the pair of transmitting and receiving antennas, active at any given moment using control signals. The interconnection of the input port with the output ports may be done based on, at least, a 3-bit digital signal.

The RF hardware module for generating and receiving the microwave signals may comprise a vector network analyzer (VNA) for measuring amplitude and phase of the transmission and reflection coefficients from the accessory at the end of the RF cables.

The CPU may execute acquisition, calibration, imaging and detection algorithms. The acquisition algorithms may communicate with the RF hardware module and with the control system of the antenna array to generate and receive the transmission and reflection coefficients in a synchronized manner. The calibration algorithms may determine if the accessory is functional, may compensate for differences in performance between antenna pairs due to design and manufacturing tolerances and may eliminate all the undesired effects on the transmission and reflection coefficients that are not produced by the target (polyp). The imaging algorithm may process the calibrated transmission and reflection coefficients to obtain a cross-sectional image of the colon using a radar based or an inverse scattering algorithm. The detection algorithm may generate an alert in case the image contains a polyp. The CPU may store data and manage errors. The CPU may allow to configure the system, update the system, control the access and manage the graphical interface. The CPU may integrate a machine-readable storage medium or data storage. Such medium may be any electronic, magnetic, optical, or other physical storage device that contains or stores executable instructions. Thus, the machine-readable storage medium may be, for example, Random Access Memory (RAM), an Electrically Erasable Programmable Read-Only Memory (EEPROM), a storage device, an optical disc, and the like. In some implementations, the machine-readable storage medium may be a non-transitory machine-readable storage medium, where the term “non-transitory” does not encompass transitory propagating signals. Machine-readable storage medium may be encoded with a series of instructions executable by a processor. The instructions may cause a processor to carry out any of the algorithms described in this disclosure.

The external unit may be housed in a housing which may comprise one or more of: an on/off button, a power supply or a cable connected to an electrical grid, a touch screen, an audible/visual/communication alert, and a USB port for software update or data collection. The dimensions of the housing may approximately be 46×32×16 cm.

In some examples, the accessory comprises 8 reception antennas and 8 transmission antennas. In some examples where the accessory comprises 8 reception antennas and 8 transmission antennas, the processing unit comprises the multiplexor and the accessory does not comprise any multiplexor. In some examples the accessory comprises 8 reception antennas, 8 transmission antennas and one or more multiplexors, as seen in FIG. 6.

The system 80 as seen, comprises a combination of hardware and software components. The hardware is composed by 1) a cylindrical ring-shaped acquisition device or endoscope accessory designed to be attached to the tip of a conventional colonoscope, and 2) an external unit with a microwave transceiver, a controlling and a processing unit. The acquisition device is connected via cables to the external unit. The acquisition device may contain two multiplexed or switched arrays of eight antennas organized in two rings, one containing the transmitting and the other the receiving antennas fed by microstrip lines. The arrays may present a cylindrical shape, the antennas may be assembled on a polyamide flexible printed circuit board that may contain the microstrip feeding lines and the two radiofrequency switches or multiplexors. The antenna applicator is designed to avoid interfering the optical visualization system at the tip of the endoscope while being safe for the patient and maneuverable for the endoscopist. To meet these requirements, the endoscope accessory may be designed as a cylindrical compact array attached at the tip of a conventional colonoscope.

The accessory 81 may be comprised within a capsule. Said capsule may provide a protective encapsulation coating the accessory 81 and ensuring biocompatibility, protection of a patient's mucosa, electrical safety, fluid tightness, a sterilizable material and resistance to the passage of time. Since it is convenient that the front part of the distal end of the endoscope is not obstructed since it is where the passage channel and the cameras are located, the encapsulation may be designed in 2 parts: an inner casing and an outer casing as seen in FIG. 9.

FIG. 9 shows an accessory 91, an outer casing 92 and an inner casing 93. The inner casing 93 is in the form of a cylindrical ring. The accessory 91 may be inserted onto outer casing 92 and, in use, a colonoscope tube may be inserted through the inner casing 93. The purpose of the inner casing 93 may be to provide a solid or non-flexible base for the first flexible substrate of the accessory 91 so that it can be rolled up and adapted to an endoscope. The inner casing may be made of a biocompatible resin. A fixing means, for example, a silicone or rubber coating, or an o-ring, is included in the inner casting allowing a good adaptation and grip on the endoscope to prevent it from moving or becoming detached during the examination. The outer casing 92 may be made of a heat-shrinkable material such as polyolefin for temporary in vivo applications or biocompatible resin. The accessory 91 may be coated with an insulating material which could be a biocompatible resin in order to ensure that moisture from a colon does not damage the circuitry and electrical safety. The wiring and tubing assembly of the endoscope may be covered with a biocompatible plastic sleeve or cover during examination, ensuring that the wiring does not injure the mucosa of a patient. The plastic sleeve may be fixed with a biocompatible tape around the outer casing. In FIG. 9 the reference 94 represents an indentation to let a space housing the antennas of the accessory 91 and the reference 95 represents a possible window for making room for the second substrate comprising each pair of antennas, and/or letting transmission lines be passed through them.

An example implementation may comprise a cylindrical ring-shaped accessory device attached to a distal end of a colonoscope. The colonoscope may comprise a flexible tube of around 13 mm in diameter configured for being inserted into a patient's colon through the rectum.

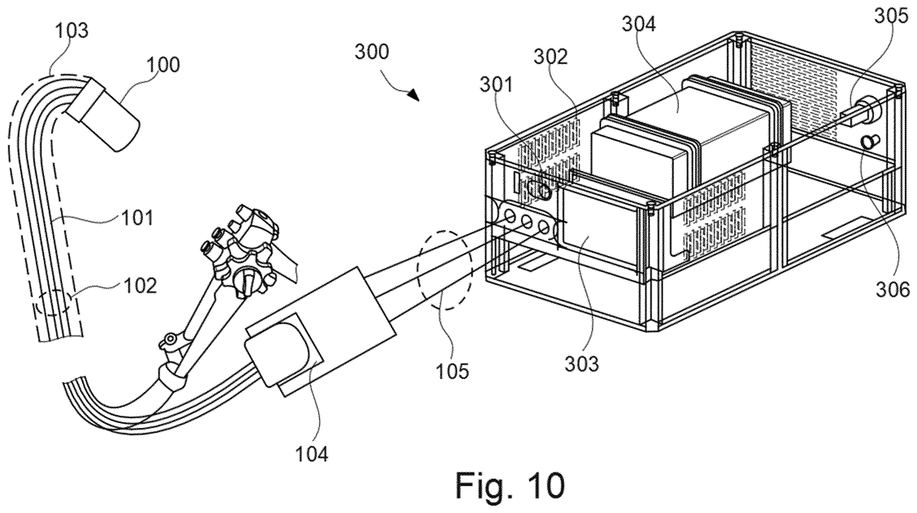

An example system is presented in FIG. 10 showing an accessory 100, an endoscope tube 101, a first group 102 of coaxial cables, which in the FIG. 10 comprises 4 coaxial cables, but the group of coaxial cables may for example comprise 16 coaxial cables, a sleeve 103 which is represented by dotted lines, a connection point 104, a second group 105 of coaxial cables and bus type cables, and an external unit 300. The external unit 300 comprises an on/off button 301, an encapsulation box 302, an LCD screen 303, a microwave transducer 304, a USB port 305 and connection means 306. The System shown in FIG. 10 comprises elements which may be disposable, such as the accessory 100, the endoscope tube 101, the first group 102 of coaxial cables, and the sleeve 103. In examples the first flexible substrate may comprise 2 layers: a top layer being a ground plane and a bottom layer comprising transmission lines. Printed circuits therein may comprise: an antenna array comprising rectangular milled windows where the antenna components are to be soldered, 2000 mm-lengthed transmission lines, and connectors. In FIG. 10, one or more of the accessory 100, and/or the endoscope tube 101, and/or the first group 102 of coaxial cables, and/or the sleeve 103 and/or the connection point 104, and/or a second group 105 of coaxial cables and/or bus type cables may be disposable or of a single use or not reusable.

A method of manufacture of an accessory according to any example comprises:
providing a first flexible substrate;
cutting at least one window on the first flexible substrate, the window defining an opening between a first side of the first flexible substrate and a second side of the first flexible substrate; the window may be cut from the first flexible substrate;
including at least a radiating element on the second substrate; including may comprise printing a radiating element on the second substrate;
attaching at least one second substrate, the second substrate comprising the at least one radiating element, to the first flexible substrate, in such a way that the at least one radiating element is at least partially aligned with the at least one window; attaching may comprise welding, or soldering;
coupling the radiating element to a transmission line; the transmission line may be comprised on the first flexible substrate.

An example of the method of manufacture comprises, before the step of soldering the second substrate:
metallizing at least an edge of the at least one window of the first flexible substrate; this step may comprise metallizing 4 edges of the window; and
metallizing the second substrate.

In some examples the method further comprises drilling via-holes on the second substrate, for example 0.6 mm via-holes, and metallizing the via-holes. Some examples further comprise metallizing the edges of the second substrate.

In some examples the method further comprises drilling via-holes on the second substrate and metallizing the via-holes.

In some examples the method further comprises printing one or more transmission lines on the first flexible substrate.

In some examples the method further comprises printing a radiating element on the second substrate before the step of soldering the second substrate on the first substrate. Printing may comprise techniques such as laser-printing, and/or photolithography, and/or screen-printing.

The preceding description has been presented to illustrate and describe certain examples. Different sets of examples have been described; these may be applied individually or in combination, sometimes with a synergetic effect. This description is not intended to be exhaustive or to limit these principles to any precise form disclosed. Many modifications and variations are possible in light of the above teaching. It is to be understood that any feature described in relation to any one example may be used alone, or in combination with other features described, and may also be used in combination with any features of any other of the examples, or any combination of any other of the examples.

What is claimed is:

1-15. (canceled)

16. An endoscope accessory, the accessory comprising:
a first flexible substrate comprising a first side and a second side, wherein the first flexible substrate further comprises a window, the window defining an opening between the first side of the first flexible substrate and the second side of the first flexible substrate;
a second substrate comprising a first surface and a second surface, the first surface of the second substrate comprising at least one radiating element, the radiating element coupled to a transmission line, the transmission line connected to a connector; wherein the transmission line is configured to transmit electrical signals towards the radiating element;
wherein the second substrate is attached to the first flexible substrate with the first surface of the second substrate adjacent to the first flexible substrate;
and wherein the radiating element is at least partially aligned with the window of the first flexible substrate.

17. The endoscope accessory of claim 16 wherein the radiating element is printed on the second substrate.

18. The endoscope accessory of claim 16 wherein the second substrate is metallized and at least an edge of the window comprises a metallized portion.

19. The endoscope accessory of claim 16 wherein the second substrate is metallized and at least an edge of the window comprises a metallized portion and wherein the edge of the window comprises a non-metallized portion aligned with the transmission line.

20. The endoscope accessory of claims 16, further comprising at least two radiating elements.

21. The endoscope accessory of claim 16, further comprising at least two radiating elements wherein the second substrate further comprises a plurality of metallized via-holes between the at least two radiating elements.

22. The endoscope accessory of claims 16 wherein the window has a width and length comprised in a range from 0.1 λto 0.5 λ, where λ represents the length of a wavelength of a microwave signal which, in use, is transmitted and/or received by the accessory.

23. The endoscope accessory of claim 16 further comprising at least two radiating elements and further comprising a multiplexor to select a signal coming from one of the at least two radiating elements.

24. The endoscope accessory of claim 16 wherein the second substrate is metallized and at least an edge of the window comprises a metallized portion and wherein the radiating element is etched on the first surface of the second substrate.

25. The endoscope accessory of claim 16 wherein the second side of the first flexible substrate comprises the transmission line and the first side of the first flexible substrate comprises a ground plane.

26. The endoscope accessory of claim 16, wherein
the radiating element is printed on the second substrate;
the second substrate is metallized and at least an edge of the window comprises a metallized portion;
and the edge of the window comprises a non-metallized portion aligned with the transmission line.

27. The endoscope accessory of claim 16 wherein
the radiating element is printed on the second substrate;
the second substrate is metallized and at least an edge of the window comprises a metallized portion;
the edge of the window comprises a non-metallized portion aligned with the transmission line; and
further comprising at least two radiating elements wherein the second substrate further comprises a plurality of metallized via-holes between the at least two radiating elements.

28. A system to detect polyps, the system comprising:
an external unit operable to generate, receive and process microwave signals and operable to activate an alert if a polyp is detected; and
and endoscope accessory, the endoscope accessory comprising:
a first flexible substrate comprising a first side and a second side, wherein the first flexible substrate further comprises a window, the window defining an opening between the first side of the first flexible substrate and the second side of the first flexible substrate;
a second substrate comprising a first surface and a second surface, the first surface of the second substrate comprising at least one radiating element, the radiating element coupled to a transmission line, the transmission line connected to a connector; wherein the transmission line is configured to transmit electrical signals towards the radiating element;
wherein the second substrate is attached to the first flexible substrate with the first surface of the second substrate adjacent to the first flexible substrate;
and wherein the radiating element is at least partially aligned with the window of the first flexible substrate.

29. The system of claim 28, further comprising one or more multiplexors, wherein
the external unit comprises the one or more multiplexors; and/or
a connection point connecting the accessory to the external unit comprises the one or more multiplexors.

30. The system of claim 28 wherein the endoscope accessory comprises the radiating element printed on the second substrate.

31. The system of claim 28 wherein the radiating element is printed on the second substrate; the second substrate is metallized and at least an edge of the window comprises a metallized portion; and the edge of the window comprises a non-metallized portion aligned with the transmission line.

32. The system of claim 28 wherein the second substrate is metallized and at least an edge of the window comprises a metallized portion and wherein the radiating element is etched on the first surface of the second substrate.

33. A method of manufacture of an endoscope accessory, the method comprising the steps of:
providing a first flexible substrate;
cutting at least one window on the first flexible substrate, the window defining an opening between a first side of the first flexible substrate and a second side of the first flexible substrate;
including at least a radiating element on the second substrate;
attaching or soldering at least one second substrate to the first flexible substrate, the second substrate comprising the at least one radiating element, in such a way that the at least one radiating element is at least partially aligned with the at least one window;
coupling the radiating element to a transmission line; and
connecting the transmission line to a connector.

34. The method of manufacture of claim 33, further comprising, before the step of soldering the second substrate, the steps of:
metallizing at least an edge of the at least one window of the first flexible substrate; and
metallizing the second substrate.

35. The method of manufacture of the claim 33, further comprising the steps of drilling via-holes on the second substrate and metallizing the via-holes.
